(19) United States
(12) Patent Application Publication
(10) Pub. No.: US 2006/0167466 A1
(43) Pub. Date: Jul. 27, 2006

(54) Intraocular lens inserter system components

(76) Inventor: Vaclav Dusek, Bellevue, WA (US)

Correspondence Address: LAW OFFICES OF RONALD M ANDERSON, 600 108TH AVE, NE, SUITE 507, BELLEVUE, WA, 98004, US

(21) Appl. No.: 11/041,316

(22) Filed: Jan. 21, 2005

(51) Int. Cl. A61F 9/013

(52) U.S. Cl. 606/107

(57) ABSTRACT

Disclosed are an intraocular lens (IOL) push rod, an IOL cartridge, multiple embodiments of an IOL cartridge housing, and a method for folding an IOL for insertion into an eye during ocular surgery. The distal end of the push rod is contoured to apply force to a substantial portion of the perimeter of an IOL to advance the IOL through a bore of the cartridge. Two hinges couple flanges on the IOL cartridge to a central portion that supports an IOL when protected by a cover for an extended storage time. The IOL cartridge has a locking element that engages a cartridge housing or IOL inserter. Each cartridge housing accepts the IOL cartridge with the IOL in an unfolded state. The cartridge bore is unobstructed and is tapered to fold one side of the IOL over the other as the IOL is advanced through the bore and into an eye.

### FIELD OF THE INVENTION

The present invention generally relates to ophthalmic devices for implanting intraocular lenses (IOLs), and more specifically, to an IOL inserter system and a push rod that uniformly applies force to a substantial portion of the perimeter of an IOL in order to controllably advance it from a cartridge into an eye.

### BACKGROUND OF THE INVENTION

Cataracts occur when the normally clear lens of the eye becomes clouded, causing vision to deteriorate, because the clouding of the lens diffuses light and thus prevents the light from being properly focused on the retina. This condition is most commonly caused by the natural aging process that causes chemical changes in the natural lens of the eye. Cataracts may also develop as a result of injury to the eye, diabetes, use of steroid medications, previous eye surgery, or an inflammation of the eye. Recent statistics indicate that over one million people undergo cataract surgery each year in the United States, and the majority of these people enjoy substantially improved vision after the surgery.

Intracapsular Cataract Extraction (ICCE) was one of the first techniques developed to treat cataracts with a surgical procedure. In this procedure, a large incision was made in the eye, because the procedure involved removing the entire natural lens, as well as the entire lens capsule that holds the natural lens in place. Patients were given glasses to wear after this surgery, and they often encountered problems acclimating to the thick lenses required in the glasses. Many patients also needed to remain in bed for several days while the eye healed.

Subsequently, safer surgical techniques were developed that dramatically reduced the recovery time for patients and provided much better vision. These new procedures employ an artificial IOL. The relatively small, lightweight plastic, silicone, or acrylic IOL could be implanted permanently in the eye using a technique known as Extracapsular Cataract Extraction (ECCE). ECCE, unlike ICCE, does not involve removing the lens capsule. Instead, an incision, usually twelve millimeters (or less) in length, is made in the eye, and the natural lens is removed in one piece. Then, the IOL is inserted into the lens capsule as a replacement for the natural lens. Multiple sutures are typically used to seal the incision in the eye after this cataract surgery. The IOL thereafter provides the fixed focusing function previously provided by the natural lens, and patients should no longer have need for relatively thick glasses.

Kelman PhacoEmulsification (KPE) is another technique that was developed as a result of searching for ways to perform cataract surgery utilizing a smaller incision. An ultrasound or laser probe breaks the natural lens apart, and the fragments are aspirated from the eye through a three millimeter incision. An IOL is then inserted through this incision, which can be closed with fewer sutures than ECCE, cauterized, or allowed to heal unsutured.

The goal of achieving safer cataract surgery and reduced patient recovery time by using smaller incisions continues to drive the development of new material for soft, foldable IOLs and more precise means of implanting the artificial lens. For example, U.S. Pat. No. 5,582,613 (Brady) discloses apparatus and methods for inserting foldable IOLs into the eye. The apparatus includes an inserter that comprises a load chamber, an injection tube, a hand piece, and a push rod. The load chamber includes first and second members that move relative to each other, to open or close the load chamber. When the first and second members are hinged away from each other so that the load chamber is open, the IOL can be received in an unfolded state between the first and second members. However, because there is only a single hinge between the first and second members, a relatively small surface supports the IOL in its unfolded position. The limited amount of support for the flexible IOL with the first and second members in the unfolded position does not enable the unfolded IOL to be retained unfolded in this position. It is undesirable to store the flexible IOL in a folded state, since it may be damaged if not stored in essentially flat state.

In the closed position, the first and second members define at least a portion of the first lumen of the load chamber, and when the members are moved into the closed position, the IOL is folded. As shown in FIG. 1 of the Brady patent, the first and second members include ribs (not provided with a reference number) that grip opposite edges of the IOL as the first and second members are moved toward each other to fold the IOL. However, when the load chamber is closed with the IOL folded therein, these ribs project inwardly into the first lumen, thus partially obstructing the first lumen so that movement of the IOL through the first lumen and into the patient's eye can be impaired. Also, the ribs can damage the haptics (very small diameter wires that extend outwardly in an arc from opposite sides of the IOL to center it within the lens capsule) of the IOL, as the IOL travels through the first lumen.

A push rod included in an IOL inserter is sized to pass through the bore of the handpiece, the first lumen of the load chamber, and through at least a portion of the second lumen of the injection tube to advance the IOL into the patient's eye. As a result of the ribs partially obstructing the lumen, the push rod must be made smaller than the lumen, which limits the contact possible with the perimeter of the IOL by the push rod, because the push rod must pass the ribs when advancing the IOL through the lumen. Thus, the diameter of a conventional push rod in an IOL inserter is too small to make much more than a point contact with the perimeter of the IOL. Due to its small diameter, the push rod may move past the IOL without contacting its perimeter and thereby fail to advance the IOL as the push rod is moved through the lumen of the load chamber.

Furthermore, when the IOL is advanced through the lumen of the load chamber and into an eye, the push rod and the injection tube do not cooperate in any manner to enable maneuverability of the IOL in the eye.

U.S. Pat. No. 4,681,102 (Bartell) discloses an apparatus and method for inserting an IOL into an eye. The IOL is placed within a hinged load chamber having a pair of flanges such that when the flanges are moved toward each other about their hinge, the IOL is folded along its length. The load chamber is fitted into an injector cylinder that is then connected to an insertion cone. The insertion cone defines a lumen for passage of the IOL into the eye of a patient. A push rod passing through the injector cylinder is used to push the IOL through the lumen and into the eye. However, like the push rod in Brady, the push rod in Bartell contacts the perimeter of the folded IOL at only a single point when advancing the IOL through the lumen. Similarly, the single hinge joining the hinges does not enable a substantial portion of the unfolded IOL to be supported in a relatively flat position.

U.S. Pat. No. 6,143,001 (Brown) discloses a lens injector cartridge having an asymmetric bore. As shown in FIG. 10 of Brown, the proximal end of the cartridge may contain a peg around which one of the haptics is looped in order to prevent the haptic from being caught by the push rod (not shown in Brown) used for advancing the IOL down the bore. However, the haptic might still be damaged as the IOL is moved distally through the bore, if the attached haptic interacts with the push rod as the haptic is unlooped from the peg while being advanced by the push rod.

Therefore, it would be desirable to provide an IOL cartridge system and push rod that enables an IOL to be controllably advanced distally through a cartridge without obstruction caused by intruding ribs and using a contoured shape on the distal end of the push rod that contacts the IOL along a substantial portion of its perimeter, protects the haptics of an IOL from damage, provides for maneuvering the IOL by grasping one of the haptics after the IOL exits the cartridge, enables a preloaded IOL to be supported in an unfolded position for long periods of time without damage, and which is optionally attached to an IOL inserter. Furthermore, the IOL cartridge system (or portions thereof) should be either disposable or reusable.

### SUMMARY OF THE INVENTION

Accordingly, one aspect of the present invention is directed towards an IOL push rod that is configured for use with an IOL inserter and employed to controllably advance an IOL disposed inside an IOL cartridge along a longitudinal axis of the IOL cartridge. The intraocular lens push rod has a proximal end against which a force is applied to advance an IOL along the longitudinal axis. The disposable push rod comprises a material that is sufficiently rigid so that the push rod does not buckle when applying the force to advance the IOL. The push rod also includes a distal end (a push rod tip) contoured to contact a substantial portion of a perimeter of a proximal end of an IOL disposed inside an IOL cartridge. The contour of the distal end of the push rod is shaped to substantially correspond to a proximal end of a folded IOL. Thus, contact with a substantial portion of a perimeter of the proximal end of an IOL by the contoured leading edge of the distal end ensures that a force is more evenly applied by the push rod to advance an IOL along the longitudinal axis of an IOL cartridge, preventing damage to the IOL. A notch is optionally provided disposed adjacent to the distal end. The notch is sized to engage a haptic of an IOL between the notch and a recess formed on a distal end of an IOL cartridge, to enable an IOL to be maneuvered inside a lens capsule of a patient's eye.

The distal end of the push rod also includes a groove that is disposed on an outer surface of the push rod and is sized to accommodate a haptic of an IOL. The distal end of the push rod is sized so that an outer surface of the distal end of the push rod is in sliding contact with a substantial portion of an inner surface of an IOL cartridge, and the distal end of the push rod cannot be advanced past a proximal end of an IOL. Furthermore, the distal end of the push rod is shaped to engage an IOL cartridge in a predefined rotational orientation.

Another aspect of the present invention is directed towards an IOL cartridge system that is configured for use with both a disposable and reusable IOL inserter and in which an IOL is pre-loaded, sterilized, and disposed prior to insertion through the cartridge and into an eye of a patient. The cartridge preferably comprises a plastic material that is disposable. The cartridge has a distal portion, a lens loading portion, a locking element, a first flange, and a second flange. There are four embodiments of a housing for the cartridge disclosed below, wherein a first embodiment of the housing cartridge comprises a permanent part of the inserter, and a second, a third, and a fourth embodiment of the housing cartridge is each adapted to be attached to a distal end of an IOL inserter.

The distal portion of the cartridge includes a first internal bore with a proximal end and a distal end. The distal end has an opening through which a folded IOL is ejected into an eye. As noted above, a leading edge of the distal end of the first internal bore also includes a recess sized to cooperate with the distal end of a push rod in grasping a haptic of an intraocular lens, to enable maneuvering the intraocular lens inside a lens capsule of a patient's eye. The lens loading portion has a central groove extending longitudinally. The locking element is disposed on the cartridge, spaced apart from the distal portion and comprises either a locking element disposed adjacent to the proximal end of the lens loading portion for removably retaining the cartridge in the first embodiment of the cartridge housing, or a first notch and a second notch disposed on the first flange and the second flange, respectively, for retaining the cartridge in any of the second, third, and fourth embodiments of the housing cartridge.

The first flange is coupled to one side of the lens loading portion by a first hinge that extends longitudinally along one side of the central groove. The second flange is similarly coupled to an opposite side of the lens loading portion by a second hinge that extends longitudinally along an opposite side of the central groove from the first hinge. The first hinge and the second hinge respectively enable rotation of the first flange and the second flange towards each other. Prior to their rotation toward each other to fold the lens, the first flange and the second flange cooperate with the lens loading portion to define a lens support region wherein an unfolded IOL is disposed (optionally for long term storage). Upon being rotated toward each other, the first flange and the second flange cause an IOL on the lens support region to be folded in preparation for its insertion through the first internal bore of the distal portion of the cartridge. One of the flanges engages the other as they are rotated into contact with each other about the first hinge and the second hinge, and the combined thickness of the first flange and the second flange is at least equal to the outer dimension of the proximal end of the first internal bore.

The first flange and the second flange each include curved surfaces extending longitudinally between their proximal and distal ends and outwardly of the first hinge and the second hinge, respectively. Inwardly projecting lips are formed and extend longitudinally along the outer edges of the curved surfaces, spaced apart from each of the first and second hinges. The lips are sized to engage diametrically opposite sides of an IOL that is supported on the lens support region, so that as the first flange and the second flange are rotated toward and into contact with each other, they form a second internal bore having a distal end and a proximal end, wherein the lips project outwardly. The lips retain an IOL as it is folded between the first flange and the second flange within the second internal bore such that during the rotation of the flanges about their respective hinges, an unfolded IOL is folded symmetrically. Additional folding also takes place as the lens is advanced into the first internal bore. The distal end of the second internal bore is in communication with the proximal end of the first internal bore and both are tapered asymmetrically and longitudinally apart to cause one side of a folded IOL to overlap an opposite side of the IOL as it is advanced through the second and first internal bores. However, the lips do not protrude into the second internal bore, leaving it free of any obstruction that can interfere with advancing an IOL through the second internal bore and into the first internal bore.

All four of the embodiments of the cartridge housing are configured to enclose at least a portion of the cartridge and include a distal end, a proximal end, a top portion and a base portion, a cartridge receiving opening, a push rod opening, and a groove. The cartridge receiving opening is disposed proximate to the distal end of the housing and is adapted to receive at least the distal portion of the cartridge. This opening engages the cartridge within the housing in a first position and enables movement of the cartridge within the housing to a second position to secure the cartridge within the housing. The push rod opening is disposed proximate to the proximal end of the housing and is adapted to receive an IOL push rod.

In the first embodiment of the cartridge housing, wherein the housing is a permanent part of the inserter, the groove is disposed in the base portion of the housing and is configured to secure the locking element included on the IOL cartridge.

The other three embodiments of the cartridge housing are attachable to an inserter and include a first opening and a second opening. In addition, the cartridge receiver opening is configured so that the first flange and the second flange are free to rotate about the first hinge and the second hinge, respectively. The first opening is disposed on the housing between the top portion and the base portion, and the second opening is disposed opposite the first opening, such that a stabilizer inserted into the first opening and the second opening is disposed substantially perpendicular to the longitudinal axis of the cartridge to prevent movement of the intraocular cartridge towards the base portion of the housing. These three embodiments of the cartridge housing also include a first protrusion and a second protrusion disposed between the top portion and the base portion of the housing. The first protrusion and the second protrusion are configured to respectively engage a first notch and a second notch included on the cartridge and to align the longitudinal axis of the cartridge with the longitudinal axis of an intraocular lens inserter such that an intraocular lens is readily advanced from the cartridge and into an eye. Also, a portion of the cartridge housing disposed between the top portion and the base portion is in sliding contact with the first flange and the second flange of the cartridge.

In the second embodiment of the cartridge housing, a distal portion of the IOL cartridge is inserted into the housing before the first flange and second flange are rotated toward each other. In this embodiment, after the first flange and the second flange are rotated toward and into contact with each other, the cartridge receiving opening enables movement of the IOL cartridge between the top portion and the base portion to seat the IOL cartridge within the housing.

In the third embodiment of the cartridge housing, the IOL cartridge is attached to the housing and pivotal within the cartridge receiving opening, from an initial position in which the first flange and the second flange are not yet rotated toward and into contact with each other, to a seated position within the cartridge receiving opening, which is attained after the first flange and the second flange have been rotated toward and into contact with each other. In the seated position, the IOL cartridge is securely locked in the position and the IOL is readily advanced from the IOL cartridge and into a patient's eye.

In the fourth embodiment of the cartridge housing, the housing is configured and adapted to be mounted to an IOL inserter so that a longitudinal axis of the distal end of the housing is not aligned with a longitudinal axis of an IOL inserter engaged by the housing when the IOL cartridge has been pivoted to the seated position, and so that the longitudinal axis passing through the first internal bore and the second internal bore of the IOL cartridge forms a predefined angle with the longitudinal axis of the IOL inserter. This angle may be a preferred angle to facilitate use of the IOL inserter and housing when inserting the IOL into the eye. In this embodiment, a flexible push rod must be employed to enable the push rod to bend as it is advanced.

The IOL cartridge system also preferably comprises a package for the IOL that is pre-loaded in the cartridge. If the first embodiment of the cartridge housing, that is permanently attached to an inserter, is to be used, the package has a base (separate from the permanently attached housing) and a cover that overlies an IOL disposed on the lens loading portion of the IOL cartridge. If any of the second, third, or fourth housing embodiments, that are attachable to an inserter, are to be used, the package includes a cover that overlies an IOL disposed on the lens loading portion of the IOL cartridge within the housing.

The cover is removable from either the base (first housing embodiment) or the cartridge housing (second, third, and fourth housing embodiments) to free the IOL cartridge for removal. Once removed, the first and second flanges are enabled to rotate towards each other to fold the IOL, and the IOL cartridge is enabled to be released from the base and inserted into the permanent housing of an IOL inserter to advance an IOL into a patient's eye. In the alternative, once the cover is removed, the first and second flanges are enabled to rotate towards each other to fold the IOL, and the IOL cartridge is enabled to move from a first position to a second position within the attachable cartridge housing so that the IOL is advanced into a patient's eye. Also, the cover secures the IOL cartridge in the open position for use with any of the second, third, or fourth embodiments of the cartridge housing that are attachable to an inserter, or for the base that is used with the first embodiment of the cartridge housing (to secure the IOL in place on the loading portion until the cover is removed).

Yet another aspect of the present invention is directed towards a method for folding an IOL disposed in an IOL cartridge into a folded state in which one side of the IOL overlaps an opposite side, as the IOL is advanced through the IOL cartridge and into an eye. With an IOL disposed between opposite flanges that are rotatable toward each other about two spaced apart hinges, the two flanges are rotated toward each other, causing the one side and the opposite side of the IOL that is initially unfolded, to be folded toward each other and forming a bore through which the IOL can be advanced after being thus folded. The bore is tapered into an asymmetrical shape, wherein a contour of a curvature of one side of the bore becomes increasingly closer in matching a final shape of the bore at the distal end of the bore. Another step includes using the push rod to force the lens that is folded distally down the bore. As the lens advances, the tapered and asymmetrical shape of the distal end of the bore forces said one side of the lens to be folded into the bore. And further distally down the bore, a tapered and asymmetrical shape of the other side of the bore forces the other side of the lens to increasingly overlap the first side of the lens as the IOL moves distally through the bore toward its distal end.

Yet another aspect of the present invention is directed towards a method of grasping and manipulating an intraocular lens that has been forcibly expelled from a cartridge and into a lens capsule of an eye such that the intraocular lens may be positioned or oriented as desired within the lens capsule using the same instrument used to insert the IOL. After the intraocular lens has been expelled into the lens capsule from the cartridge using a push rod of an intraocular lens inserter, the user positions a notch formed in the distal end of the push rod and the surface of the leading edge of the cartridge so as to grasp a haptic of the intraocular lens as the user retracts the push rod of the intraocular lens inserter. Finally, the user maneuvers the haptic of the intraocular lens into a desired position or orientation within the lens capsule of the eye.

### BRIEF DESCRIPTION OF THE DRAWING FIGURES

The foregoing aspects and many of the attendant advantages of this invention will become more readily appreciated as the same becomes better understood by reference to the following detailed description, when taken in conjunction with the accompanying drawings, wherein:

FIG. 1A is a cross-sectional side view of an IOL inserter, with an IOL cartridge and IOL push rod in accord with the present invention;

FIG. 1B is a top view of the distal portion of the IOL inserter of FIG. 1A shown without the IOL cartridge;

FIG. 2A is a side view of an IOL push rod in accord with the present invention;

FIG. 2B is a bottom view of the IOL push rod of FIG. 2A;

FIG. 2C is a cross-sectional view of the IOL push rod of FIG. 2A, taken along section line 2C-2C in FIG. 2A;

FIG. 2D is a side view of an alternative IOL push rod in accord with the present invention;

FIG. 3A is a top view of an IOL cartridge in accord with the present invention, shown with the flanges in an open position;

FIG. 3B is a transverse view of the IOL cartridge of FIG. 3A;

FIG. 3C is a cross-sectional view of the IOL cartridge of FIG. 3A, taken along section line 3C-3C of FIG. 3B;

FIG. 3D is a transverse view of the IOL cartridge of FIG. 3A, showing the flanges in a closed position;

FIG. 3E is a side view of the IOL cartridge of FIG. 3A, showing the flanges in a closed position;

FIG. 3F is a cross-sectional view of an alternative IOL cartridge;

FIG. 4A is a top view of an IOL cartridge in accord with the present invention, shown with the flanges in an open position and shown supporting an unfolded IOL;

FIG. 4B is a transverse view of the IOL cartridge of FIG. 4A;

FIG. 4C is a transverse view of the IOL cartridge of FIG. 4A, shown with the flanges in a closed position, so that the previously unfolded IOL has been now folded;

FIG. 5A is a side view of the IOL cartridge with the flanges in an open position and disposed in an initial position within a second embodiment of an IOL cartridge housing;

FIG. 5B is a cross-sectional view of the IOL cartridge shown in FIG. 5A, taken along section line 5B-5B in FIG. 5A;

FIG. 5C is a side view of the IOL cartridge with the flanges in a closed position and seated within the second embodiment of the IOL cartridge housing;

FIG. 5D is a cross-sectional view of the IOL cartridge of FIG. 5C, taken along section line 5D-5D in FIG. 5C;

FIG. 6A is a top view of an IOL package cover;

FIG. 6B is a side view of the IOL package cover of FIG. 6A;

FIG. 7A is a side view of the IOL package cover of FIGS. 6A and 6B, shown attached to the cartridge housing of FIG. 5A;

FIG. 7B is a side view of the IOL package cover of FIGS. 6A and 6B, shown attached to an IOL package base;

FIG. 8A is an exploded cross-sectional side view of an IOL inserter and an IOL cartridge housing of FIG. 5A (shown without the IOL cartridge for clarity);

FIG. 8B is a side view of the IOL cartridge housing of FIG. 5A shown attached to an IOL inserter wherein the IOL cartridge includes an unfolded IOL;

FIG. 8C is a side view of the IOL cartridge housing of FIG. 5A shown attached to the IOL inserter of FIG. 8B, wherein the IOL cartridge includes a folded IOL;

FIG. 9A is a side view of the IOL cartridge with the flanges in an open position and shown disposed in an initial position within a third embodiment of an IOL cartridge housing;

FIG. 9B is a cross-sectional view of the IOL cartridge and cartridge housing of FIG. 9A, taken along section line 9B-9B in FIG. 9A;

FIG. 9C is a side view of the IOL cartridge with the flanges in a closed position and disposed in a seated position within the third embodiment of the IOL cartridge housing;

FIG. 9D is a cross-sectional view of the IOL cartridge and cartridge housing of FIG. 9C taken along section line 9D-9D in FIG. 9C;

FIG. 10A is an exploded cross-sectional side view of an IOL inserter shown with the IOL cartridge housing of FIG. 9A (shown without the IOL cartridge for clarity);

FIG. 10B is a side view of the IOL cartridge housing of FIG. 9A, shown attached to an IOL inserter with the flanges of the cartridge in an open position;

FIG. 10C is a side view of the IOL cartridge housing of FIG. 9A shown attached to an IOL inserter with the flanges of the cartridge in a closed position;

FIG. 11A is a side view of the IOL cartridge with the flanges in an open position and disposed in an initial position within a fourth embodiment of an IOL cartridge housing;

FIG. 11B is a cross-sectional view of the IOL cartridge and IOL cartridge housing of FIG. 11A, taken along section line 11B-11B shown in FIG. 11A;

FIG. 11C is a side view of the IOL cartridge with the flanges in a closed position and disposed in a seated position within the fourth embodiment of the IOL cartridge housing;

FIG. 11D is a cross-sectional view of the IOL cartridge and IOL cartridge housing of FIG. 11C, taken along section line 11D-11D shown in FIG. 11C;

FIG. 12A is an exploded cross-sectional side view of an IOL inserter shown with the IOL cartridge housing of FIG. 11A (shown without the IOL cartridge for clarity);

FIG. 12B is a side view of the IOL cartridge housing of FIG. 11A, shown attached to an IOL inserter with an IOL cartridge in the open position;

FIG. 12C is a side view of the IOL cartridge housing of FIG. 11A shown attached to an IOL inserter with the IOL cartridge in a closed position;

FIG. 13A is a cross-sectional side view of the alternative IOL push rod of FIG. 2D and the alternative IOL cartridge of FIG. 3F, positioned to grasp an IOL haptic; and

FIG. 13B is a cross-sectional side view of the alternative IOL push rod and alternative IOL cartridge of FIG. 13A, shown grasping an IOL haptic (only the cross section of which is shown to simplify the view).

### DESCRIPTION OF THE PREFERRED EMBODIMENT

Overview of the Present Invention

The present invention includes two embodiments of an IOL push rod that more evenly apply force to a perimeter of an IOL to advance it into a patient's eye, the second embodiment of the IOL push rod being configured to grasp an IOL haptic to enable maneuvering the IOL after the IOL is in the patient's eye, an IOL cartridge system that addresses several problems found in prior art IOL cartridges, including an alternative cartridge that cooperates with the second IOL push rod embodiment to enable an IOL haptic to be grasped so that an IOL can be maneuvered once the IOL is in the patient's eye, and four embodiments of an IOL cartridge housing for use with the IOL cartridges. The present invention is versatile and improved over the prior art, and the components of this invention may be either reusable or disposable. Also, each of the IOL cartridge housings are quickly and easily attached to a distal portion of an IOL inserter. In addition, components of the present IOL cartridge system may be used with the IOL inserter illustrated in a commonly assigned U.S. patent application filed on Nov. 12, 2004, Ser. No. 10/987,351, entitled “INTRAOCULAR LENS INSERTER,” the entire specification and drawings of which are hereby specifically incorporated herein by reference, thereby ensuring a complete system for inserting an artificial IOL into a patient's eye during ocular surgery. In addition, the components enable an IOL to be stored in an unfolded state for a relatively long period of time, while being protected from damage so that minimal steps are required prior to use of the present invention during an ocular surgical procedure.

In order to protect the unfolded IOL disposed within the cartridge until it is ready to be used in a surgical procedure, a package is provided that includes a package cover overlying an opened cartridge with an unfolded IOL; the cartridge is attached to a base of the package. The package serves to protect the unfolded IOL and to prevent movement of the unfolded IOL and the cartridge in which the IOL is disposed, for instance, during transit from a supplier and during its shelf life prior to use. A view that is generic to the different embodiments of the cartridge housing, with the package cover attached to the cartridge housing and unfolded cartridge, is shown in FIG. 7A. The cartridge housing preferably includes a cartridge with an unfolded IOL disposed therein. In this option, quick removal of the package cover and completion of minimal steps, which are discussed in detail below, ensure the efficient use of the present invention during an ocular surgery.

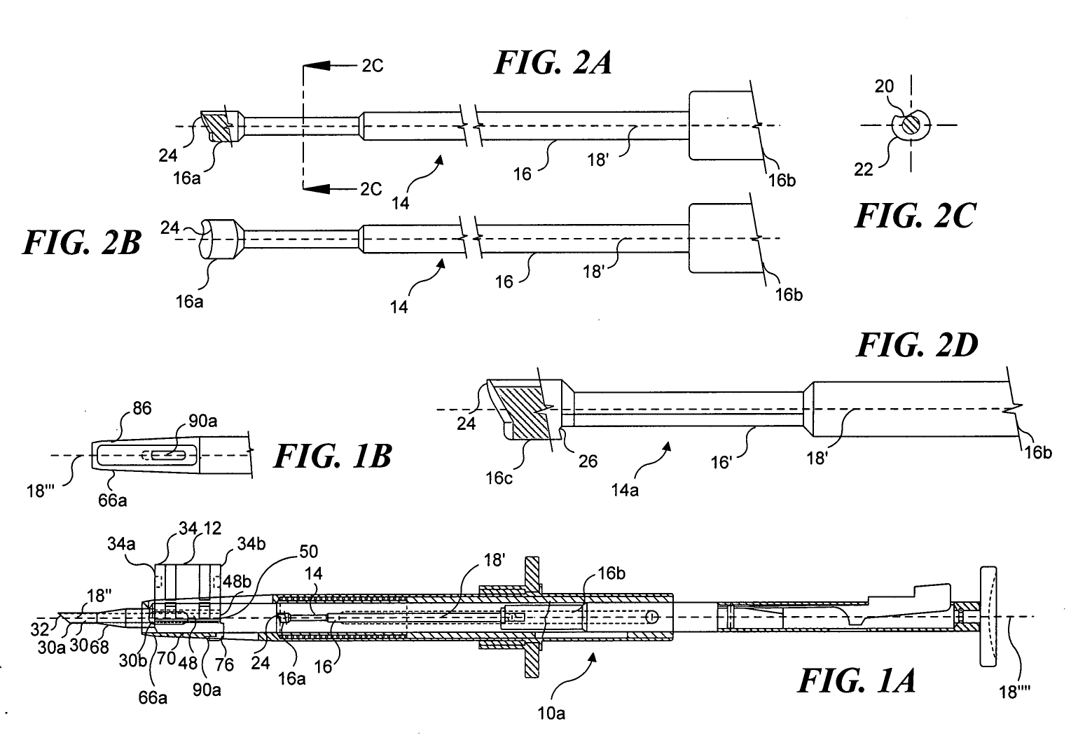

Alternatively, the package base may be used to protect only a cartridge disposed within that includes an unfolded IOL intended for use in an IOL inserter that does not employ a housing to secure the IOL cartridge to the distal end of the IOL inserter. Specifically, FIG. 1A illustrates an IOL inserter 10a (from the above-noted patent application that is incorporated herein by reference) being utilized with components of the present invention, including a IOL push rod 14 and an IOL cartridge 12. Note that this IOL inserter does not have a cartridge housing that is attached thereto. Accordingly, the package cover used for protecting the IOL is not removed from a cartridge housing, but is instead removed from a package base that is holding the cartridge. Then, the cartridge, including the unfolded IOL is closed, thus folding the IOL. Then the cartridge and folded IOL are removed from the package base and are placed directly into a distal end 66a of the IOL inserter as described in more detail below, in connection with FIG. 1A and 1B.

A number of different combinations of the disposable and reusable components comprising the present invention can be used in connection with an IOL inserter system, so the following examples are not intended to be limiting on the scope of this invention. To illustrate, in FIGS. 1A and 1B, since a separate cartridge housing is not required for IOL inserter 10a, the inserter is likely to be reused with a succession of disposable cartridges 12 that are preloaded with an unfolded IOL. Thus, after use in one surgical procedure, the disposable cartridge that is now emptied of its IOL is removed from inserter 10a, and the IOL inserter is cleaned and sterilized so that it can be used with a different IOL cartridge and IOL. In the alternative, inserter 10a may be a disposable inserter such that after use in one surgical procedures, the disposable cartridge and inserter are both disposable.

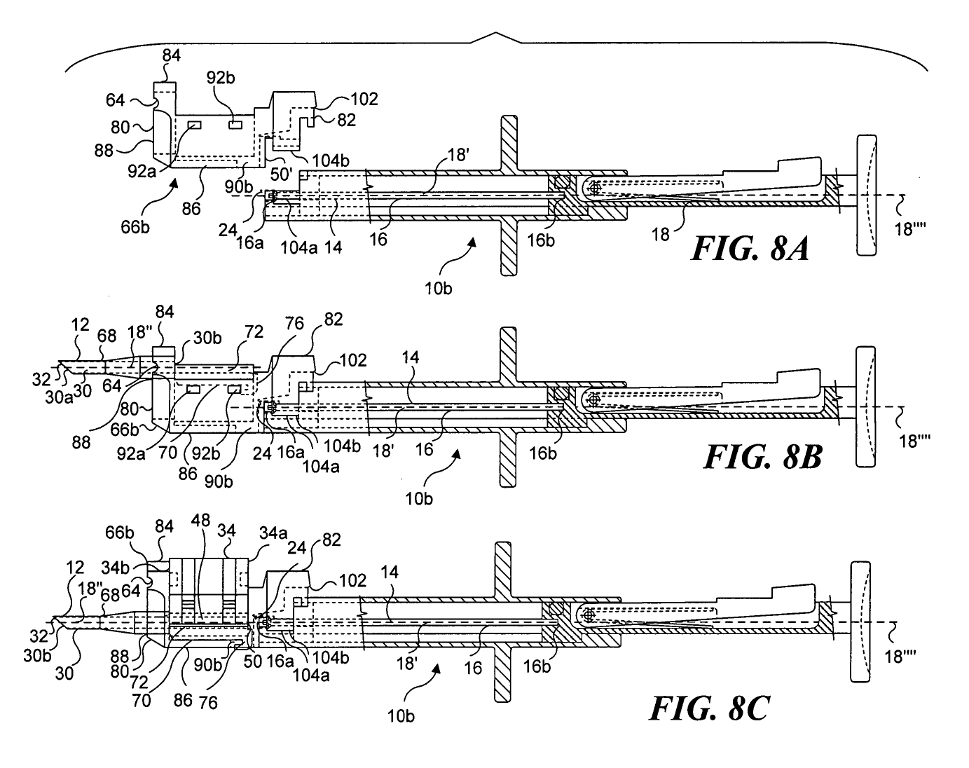

However, cartridge housing 66b, cartridge housing 66c, and cartridge housing 66d that attach respectively to IOL inserters 10b, 10c, and 10d, which are respectively shown in FIG. 8B, 10B, and 12B, are all intended to be disposable. Thus, after use in a surgical procedure, the disposable IOL cartridge that is now emptied of an IOL and the disposable cartridge housing that is attached to inserters 10b, 10c, and 10d along with the inserters will be discarded.

IOL Push Rods

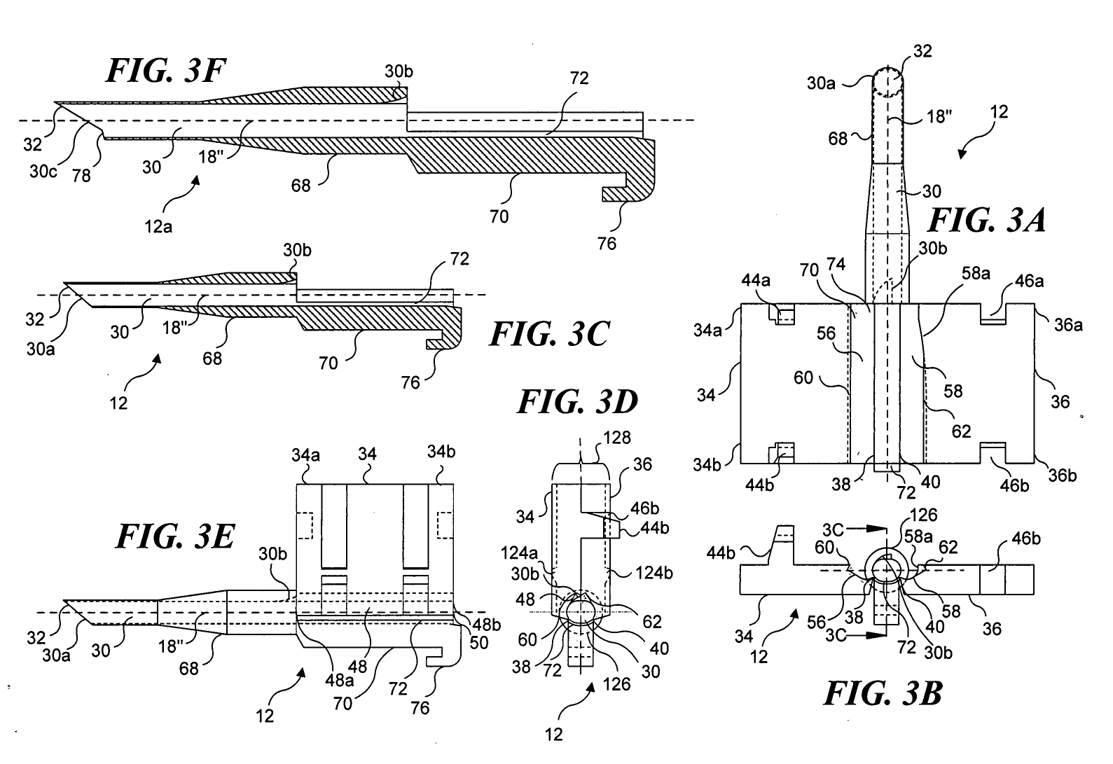

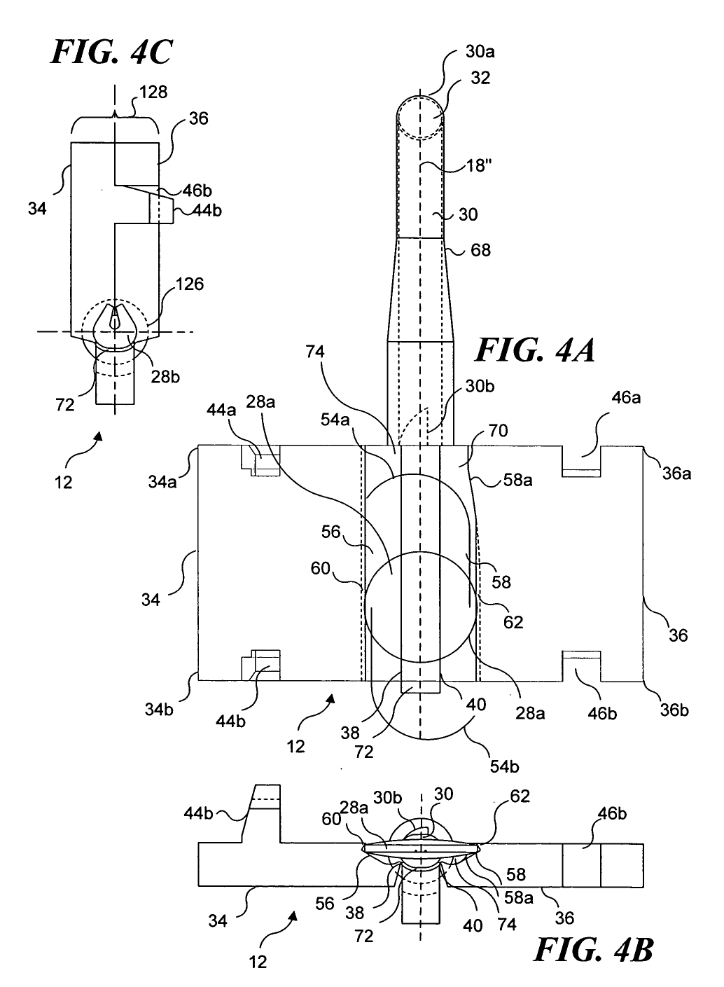

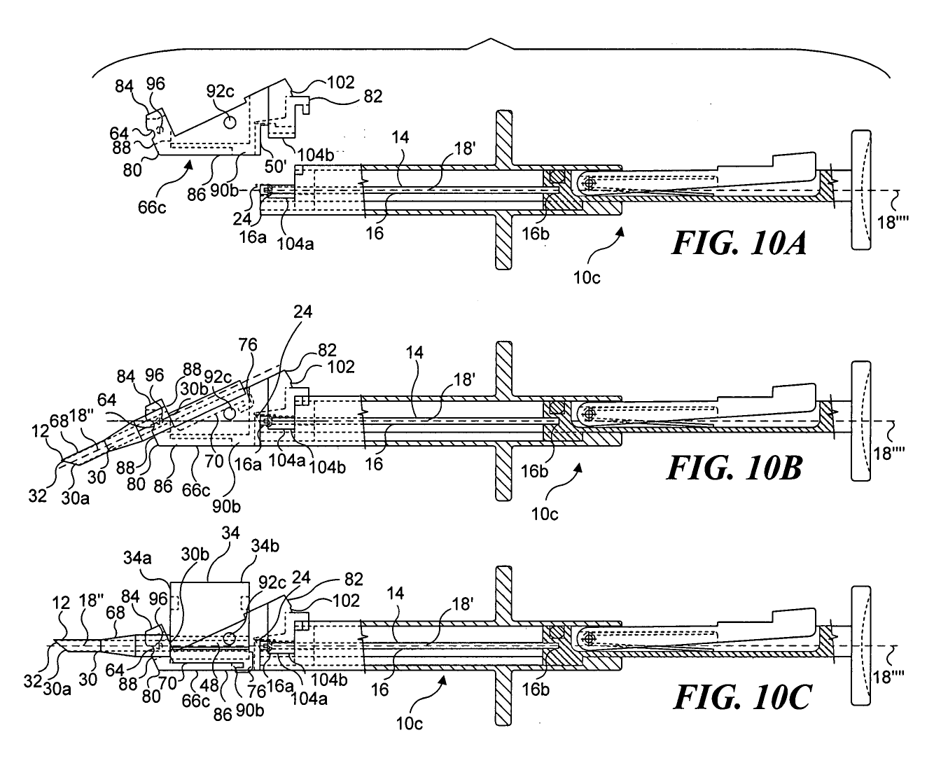

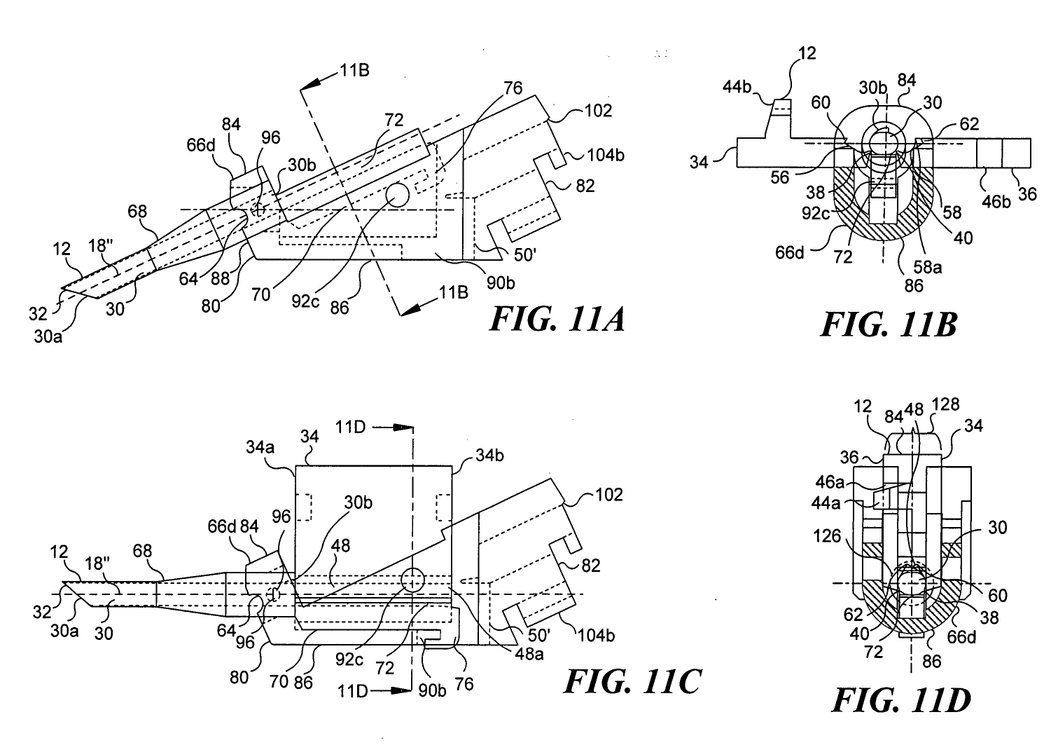

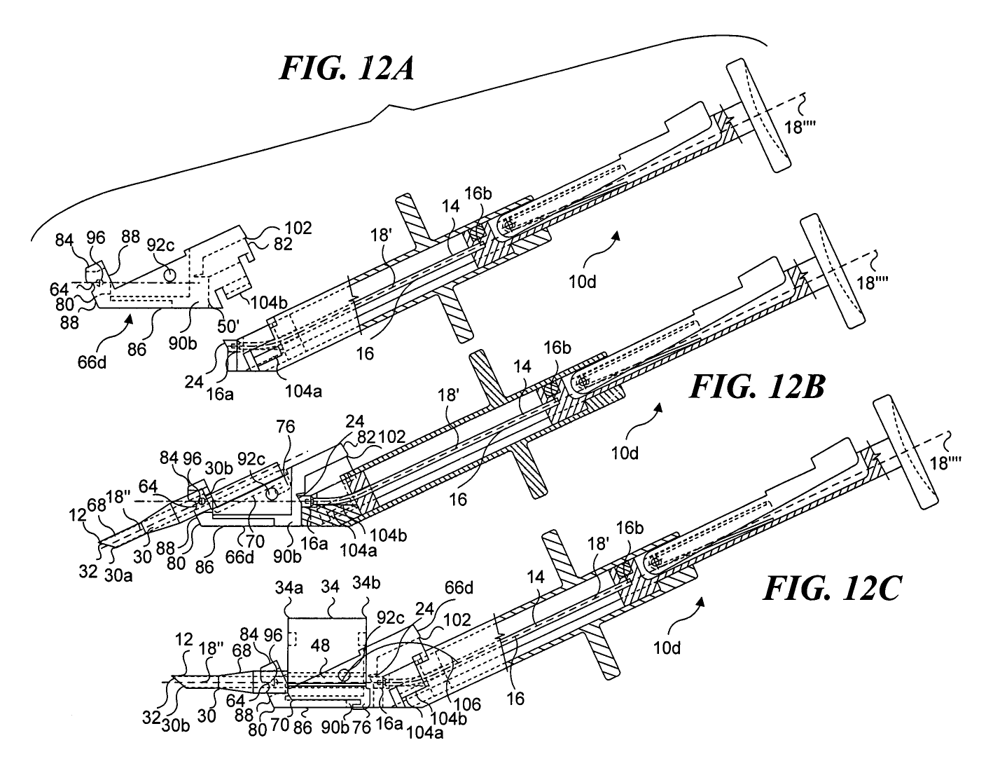

FIGS. 2A, 2B, and 2C illustrate an IOL push rod 14, which is one component of the present invention. FIG. 2D illustrates an alternative embodiment of an IOL push rod 14a. IOL push rod 14 comprises a push rod 16 having a distal end 16a and a proximal end 16b, a longitudinal axis 18′, a haptic groove 20, an outer surface 22, and a contoured leading edge 24 that is formed on the distal end of the push rod. IOL push rod 14a is similar to push rod 14, except that a push rod 16′ on IOL push rod 14a has a distal end 16c that includes a notch 26, the purpose of which is discussed below. Push rod 14 and push rod 14a are configured to advance an IOL distally of longitudinal axis 18″″ of IOL inserters 10a (FIG. 1A), 10b (FIG. 8C), 10c (FIG. 10C), or 10d (FIG. 12C) in response to a manual force (not shown) that is applied at the proximal end. Distal end 16a and distal end 16c of push rods 16 and 16′ are contoured so that when they make contact with a folded IOL 28b (FIG. 4C) in cartridge 12 (FIG. 3E) or cartridge 12a (FIG. 3F) respectively, contoured leading edge 24 (FIG. 2A and FIG. 2D) of distal end 16a and distal end 16c contacts a substantial portion of a proximal side of the perimeter of folded IOL 28b. The push rod is sized and shaped to contact the IOL while in a predefined rotational position about the longitudinal axis of the IOL cartridge.

In addition, as also illustrated in FIG. 2C, distal end 16a and distal end 16c include haptic groove 20 formed in outer surface 22. Haptic groove 20 extends from contoured leading edge 24 a sufficient distance proximal back from contoured leading edge 24 such that a proximal haptic 54b (FIG. 4A) of the folded IOL (FIG. 4C) readily fits within the haptic groove. The outer surface of the distal end of the push rod is sized sufficiently large that it remains in sliding contact with a substantial portion of the inner surface of the IOL cartridge which prevents the distal end of the push rod from advancing past the proximal end of the IOL, as can happen with prior art push rods that are substantially smaller in diameter than the bore through which they advance the IOL. The larger cross-sectional size of IOL push rods 14 and 14a is friendlier to the IOL, because it cannot slip past the IOL and damage it, and because it also ensures that force is more uniformly applied to the IOL to advance the IOL distally in a manner so that a final desired folded shape of the folded IOL is achieved. As noted above, smaller incisions in the eye are desired when performing cataract surgery, and the shape and smaller size of the folded IOL determines the incision size. Accordingly, it is clearly more desirable to provide a smaller diameter of folded IOL for insertion into the eye through an incision.

Furthermore, without the haptic groove, haptic 54b might be crushed or otherwise damaged when the folded IOL is being advanced distally by the push rod, since the clearance between the outer surface of the distal end of the push rod and the inner surface of the cartridge is minimal. The presence of haptic groove 20 ensures that the haptic does not have any damaging force inadvertently applied by the push rod and is protected in the haptic groove as the IOL is longitudinally advanced.

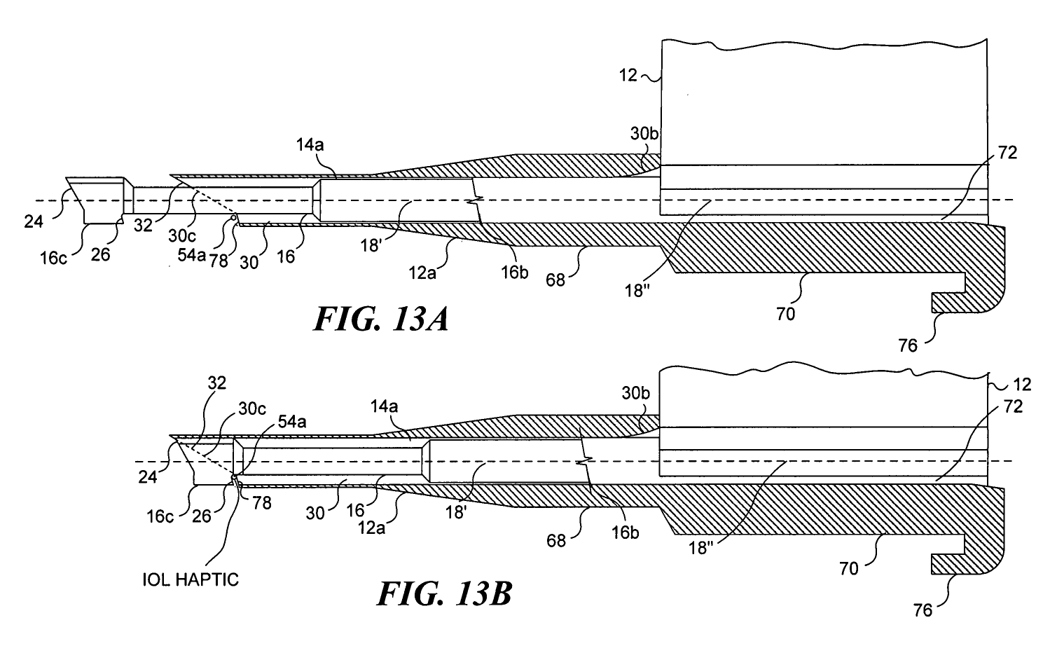

As illustrated in FIG. 2D, push rod 14a includes notch 26 disposed on the trailing edge of distal end 16c . Notch 26 is sized to provide for engaging or grasping the haptic of an IOL after the IOL has been inserted into an eye. When push rod 14a is aligned with cartridge 12a (FIG. 3F), the haptic of the IOL is trapped between notch 26 and a recess 78 on the distal end of cartridge 12a, in order to enable a user to maneuver the IOL in the lens capsule of a patient's eye. These details are described below in connection with FIGS. 13A and 13B.

Those skilled in the art will realize that distal ends 16a and 16c of the IOL push rod may comprise a disposable material such as plastic, while the rest of the push rod or other portions of the push rod comprise either a reusable material, such as a metal, or a disposable material, such as a plastic. In the alternative, the distal end of the push rod may comprise a metallic material that is reusable, while the rest of the push rod comprises a disposable material, such as a plastic. Regardless of the material selected, the push rod comprises a material that is sufficiently stiff so as to not buckle, as a force is applied to move the folded IOL distally through the IOL inserter.

Exemplary IOL Cartridges

FIG. 3A, FIG. 3B, and FIG. 3C illustrate an exemplary preferred embodiment of IOL cartridge 12, which is another component of the present invention. FIG. 3F illustrates an alternative embodiment of IOL cartridge 12a. In all of these Figures, IOL cartridges 12 and 12a are illustrated in an open state, in which they support an unfolded IOL (however, the unfolded IOL is not shown for clarity purposes). In contrast, FIG. 4A and FIG. 4B also illustrate IOL cartridge 12 in an open state, but include an unfolded IOL 28a for illustrative purposes. Note that cartridge 12 and cartridge 12a, unlike prior art cartridges, do not require an IOL to be placed in the cartridge because cartridge 12 and cartridge 12a are pre-loaded with an unfolded IOL that is disposed in its proper position within the cartridges and is ready to be folded. Thus, these cartridges eliminate the extra step wherein a user obtains an unfolded IOL from another source and positions the unfolded IOL in its proper position in the cartridge. In addition, FIG. 3D and FIG. 3E illustrate IOL cartridge 12 in a closed state, but without an folded IOL. In contrast, FIG. 4C illustrates IOL cartridge 12 in a closed state and illustrates how the IOL appears when it has been folded.

As shown in FIG. 3A and FIG. 4A, IOL cartridge 12 includes a distal portion 68, a first flange 34 and a second flange 36 that are disposed proximally of the distal portion, and a lens loading portion 70 that is disposed between the first flange and the second flange. Distal portion 68 includes a first internal bore 30 having a distal end 30a from which a folded IOL is ejected through an IOL ejector opening 32 and a partial chamfered proximal end 30b, and a longitudinal axis 18″. First flange 34 includes a distal end 34a and a proximal end 34b, and second flange 36 includes a distal end 36a and a proximal end 36b. The lens loading portion includes a central groove 72 that is disposed proximally of the first internal bore and extends longitudinally towards the proximal end of the IOL cartridge. First flange 34 is coupled to central groove 72 via a first hinge 38 along one side of the central groove. Similarly, second flange 36 is coupled to central groove 72 via a second hinge 40 along an opposite side of the central groove. The first hinge and the second hinge enable rotation of the first flange and the second flange toward each other. First flange 34 includes a distal protrusion 44a and a proximal protrusion 44b, and second flange 36 includes a proximal notch 46a and a distal notch 46b. FIG. 4A additionally illustrates unfolded IOL 28a with distal haptic 54a and proximal haptic 54b disposed within a lens support region 74, which is disposed between the first flange, the second flange, and above the central groove of the lens loading portion. Lens support region 74 is formed such that it is wider and provides more level support than the prior art; and as a result, it can readily support the unfolded IOL so that it may be stored within the IOL cartridge (when protected by a package cover that is described below) for a relatively long period of time before use, without any damage.

FIG. 3B and FIG. 4B respectively illustrate a transverse view of cartridge 12 without and with an IOL. First flange 34 includes a first lip 60 that extends longitudinally from proximal end 34b to distal end 34a (FIG. 3A and FIG. 4A) and is spaced apart from the first hinge by a first curve 56. The first lip projects inwardly toward the longitudinal axis. Similarly, second flange 36 includes a second lip 62 that extends longitudinally from proximal end 36b to distal end 36a (FIG. 3A and FIG. 4A) and is spaced apart from the second hinge by a second curve 58. It will be understood that the first curve and the second curve can instead be tapered flat surfaces. The second lip also projects inwardly towards the longitudinal axis of the IOL cartridge. As described below, the first curve, the second curve, and the central groove define a second internal bore 48 (FIG. 3D) when the first flange and the second flange are rotated towards each other. Also note that the distance that second lip 62 is spaced apart from second hinge 40 (FIG. 3A) varies. Specifically, as second lip 62 extends towards partial chamfered proximal end 30b of the first internal bore, the distance that the second curve separates the second lip from the second hinge diminishes to create a tapered second curve 58a that affects the size of the second internal bore. FIG. 4B additionally illustrates unfolded IOL 28a disposed in a lens loading area 70. Note that the opposite edges of the unfolded IOL are held securely in place by first lip 60 and second lip 62.

FIG. 3C illustrates that the IOL cartridge also comprises a locking element 76 disposed at the proximal end of cartridge 12 and spaced apart from the distal portion of the cartridge. Locking element 76 is used to removably secure the IOL cartridge to cartridge housing 66a, the first housing embodiment, as shown in FIG. 1A. The locking element in the preferred embodiment is illustrated as a hook, but those skilled in the art will realize that other suitable locking elements could instead be used, such as a latch or a protrusion on either cartridge housing 66a or the IOL cartridge that engages the other of the housing or cartridge. Thus, the present invention is not limited to a locking element shaped like a hook, but is intended to include any form of locking element that functions to retain the IOL cartridge within either an IOL cartridge housing or in the distal end of an IOL inserter.

FIGS. 3D and 4C illustrate cartridge 12 without and with the folded IOL, respectively, and also show the flanges in a closed state. In order to be disposed in a closed state, first flange 34 that is coupled to central groove 72 by first hinge 38 is rotated clockwise towards second flange 36, which is coupled to central groove 72 by second hinge 40 and is rotated counterclockwise. Protrusion 44b engages notch 46b to lock flanges of the cartridge together in closed state. Those skilled in the art will recognize that various other engaging means besides the protrusion and notch described above and shown in the Figures can be employed to secure the flanges together. For example, clamps, hooks, or snaps could be used for this purpose, or alternatively, the engaging means can optionally be omitted, since the IOL cartridge housing can maintain the IOL cartridge in a closed state. Therefore, the present invention is not limited to the preferred embodiment of engaging means that comprises the notch and the protrusion that engages the notch.

In addition, FIG. 3D also illustrates another locking element option, in addition to locking element 76 described above. First notch 124a and second notch 124b are disposed on the first flange and the second flange, respectively of cartridge 12. As described below in connection with cartridge housings 66b, 66c, and 66d, the first notch and the second notch snap or click into protrusions disposed on opposite walls of the cartridge housings when the cartridge is moved from its initial position to its seated position.

Also notice on FIG. 3D, unlike prior art cartridges, that thickness 128 of the first flange and the second flange shown in an engaged position is greater than the outside dimension 126 of the proximal end of the first internal bore. Note that the outside dimension is measured perpendicular to the contact plane of the flanges in a preferred embodiment. Thus, this feature ensures that the first flange and the second flange remain in sliding contact with a cartridge housing, thus eliminating rotational movement of the cartridge about longitudinal axis 18″ when the cartridge is moved from its initial position to its seated position, and subsequently preventing rotational movement of the cartridge about longitudinal axis 18″ when the push rod advances the IOL through the seated cartridge.

As a result of the rotation of the flanges that moves the first curve, the second curve, and tapered second curve 58a closer to the longitudinal axis of the IOL cartridge, second internal bore 48 is formed, as shown in FIG. 3D. Note that a base portion of the second internal bore (i.e., the portion comprising the central groove) can be ellipsoidal, circular, or oval in shape, but those skilled in the art will realize that the shape of the base portion of the second internal bore can differ from these exemplary embodiments As the second internal bore is formed, the IOL, as shown in FIG. 4A and FIG. 4B, is folded symmetrically, producing folded IOL 28b, as shown in FIG. 4C. A controlled folding of IOL 28a occurs since the first lip and the second lip (FIG. 4B) prevent the unfolded IOL from slipping out of position as the flanges are rotated about their respective hinges. The folded IOL is now ready to be further urged through the asymmetrical shape of the second internal bore and the first internal bore, as described below.

As illustrated in FIG. 3E, the rotation of the first flange and the second flange creates second internal bore 48 that has a distal end 48a and a proximal end 48b. A second bore push rod opening 50 is disposed at the proximal end 48b of the second internal bore. Distal end 48a of the second internal bore is now in communication with partial chamfered proximal end 30b of the first internal bore. Thus, when IOL push rod 14 enters second bore push rod opening 50, it contacts a substantial portion of the perimeter of the folded IOL. In a preferred embodiment, the right side of the symmetrically folded IOL that is tucked under the second lip is turned down closer to the center of the IOL as it is advanced through the second internal bore, because the second internal bore has a diminishing size due to the tapered second curve produced by the second lip. Thus, the folded IOL is now asymmetrical in shape. Then, as the IOL is further advanced into the first internal bore, because of partial chamfered proximal end 30b, the left side of the IOL that was tucked under the first lip turns over the right side of the folded IOL that was further folded by the tapered second curve of the second internal bore. Eventually the IOL is ejected through IOL cartridge opening 32 and into a patient's eye. Because the first lip and the second lip do not protrude into the second internal bore, unlike the prior art, the second internal bore is free of obstruction caused by these lips and thus, as described above, IOL push rod 14 can readily move the folded IOL through the second internal bore without interference from such obstructions.

Cartridge 12a of FIG. 3F includes all of the elements of cartridge 12 and functions in the same manner as cartridge 12, as described above, but cartridge 12a additionally includes recess 78 at a distal end 30c of the first internal bore. Instead of being sheared along a planar line like distal end 30a, which is shown in FIG. 3C, distal end 30c of first internal bore 30 on cartridge 12a includes two surfaces that form recess 78. Recess 78 is sized so that distal haptic 54a may be grasped and held in place between notch 26 and the recess.

### Second Embodiment of Cartridge Housing

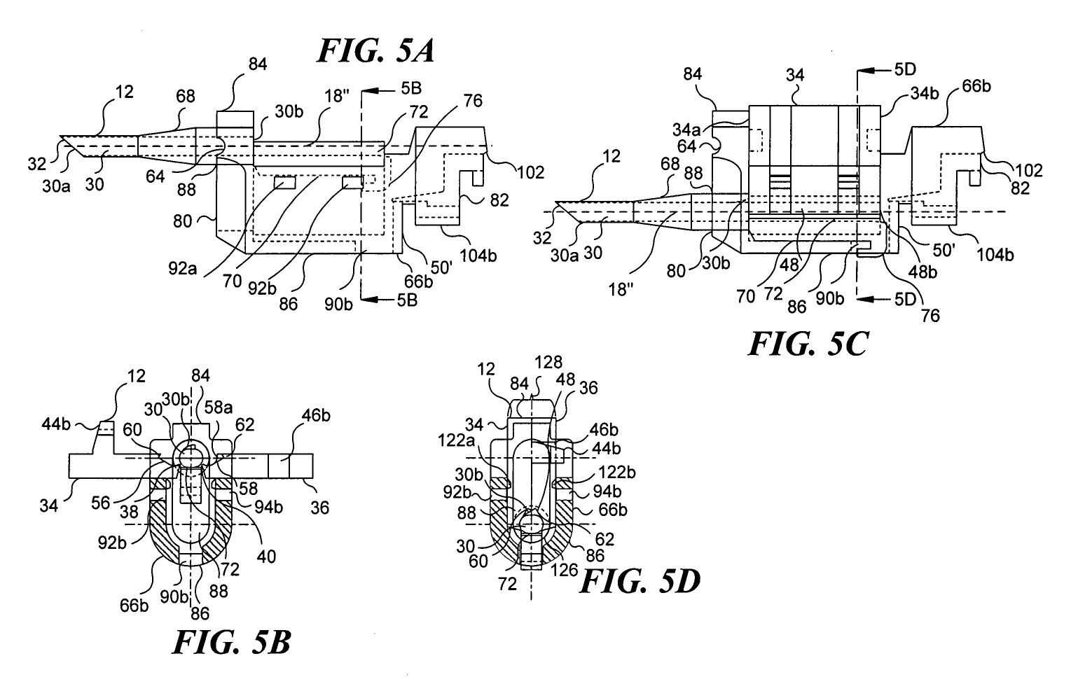

FIGS. 5A, 5B, 5C, and 5D illustrate the second of four preferred embodiments of yet another component of the present invention, a cartridge housing that is attached to the distal end of an IOL inserter, as shown in FIGS. 8B, and 8C and as shown prior to attachment in the exploded view of FIG. 8A.

As illustrated in FIG. 5A, cartridge housing 66b includes a distal end 80, a proximal end 82, a top portion 84, a base portion 86, a cartridge receiving opening 88, a housing push rod opening 50′, and a housing groove 90b. Cartridge receiving opening 88 is sized and disposed towards distal end 80 such that distal portion 68 of cartridges 12 or 12a may be inserted into cartridge receiving opening 88. (It will be understood in connection with the following discussion that cartridge 12a can be used instead of cartridge 12.) The cartridge housing encloses at least a portion of the cartridge. Also, the cartridge receiving opening 88 is sized such that the IOL cartridge is movable between the top portion and the base portion of the cartridge housing. However, first flange 34 and second flange 36 are free to rotate about first hinge 38 and second hinge 40 while the IOL cartridge is at the top portion of the cartridge housing. Housing push rod opening 50′ is disposed towards proximal end 82 and is sized to accommodate push rod 14 (FIG. 2A). FIG. 5A illustrates a first opening 92a and a second opening 92b that are disposed on the outer surface of the cartridge housing between the top portion and the base portion.

As best seen in FIG. 5B, when cartridge housing 66b has IOL cartridge 12 disposed therein, with the first flange and the second flange disposed in an open state, there is also a corresponding second opening 94b disposed on the opposite outer surface of the cartridge housing relative to where second opening 92b is disposed and between the top portion and the base portion. Although not shown, there is also a corresponding first opening 94a disposed on the opposite outer surface of the cartridge housing between the top portion and the base portion relative to where first opening 92a is disposed. In order to prevent cartridge 12 from inadvertently moving and sliding down towards the base portion of the cartridge housing, one prong of a stabilizer (not shown) can be inserted through first opening 92a (FIG. 5A) and first opening 94a and another prong of the stabilizer (not shown) can be inserted through second opening 92b and second opening 94b. These two stabilizers thus help to support cartridge 12 in its initial position as shown in FIG. 5A and when the flanges are rotated toward each other to fold the IOL, so that the cartridge is not inadvertently moved downwardly. Hence, the stabilizers are disposed substantially perpendicular to the longitudinal axis of the cartridge.

Exemplary IOL Package

Before discussing how the IOL cartridge is manipulated from its initial position (FIG. 5A) to its seated position (FIG. 5C), note that for cartridge housings 66b, 66c, and 66d that are attached to the IOL inserter, it is necessary to stabilize IOL cartridges 12 or 12a within these cartridge housings so as to ensure that there is no movement of the IOL cartridge in the cartridge housing prior to folding the IOL. Stabilization thereby ensures proper disposition of an unfolded IOL until the flanges of the IOL cartridge are rotated to fold the unfolded IOL. Stabilization of the IOL and the cartridge are ensured by package cover 100, which is attached to cartridge housings 66b, 66c, and 66d.

However, an alternative IOL package 120b is available that comprises package cover 100, a cartridge with an unfolded IOL and a package base 98b, as shown in FIG. 7B. Package 120b is intended for use with inserter 10a, which is an inserter without an attachable cartridge housing such as cartridge housing 66a. As described above, after being used during an ocular surgery, inserter 10a may be sterilized and reused. Thus, the need arises for a safe shipping technique for a the cartridge with a pre-loaded IOL. Sterilized package 120b serves that purpose.

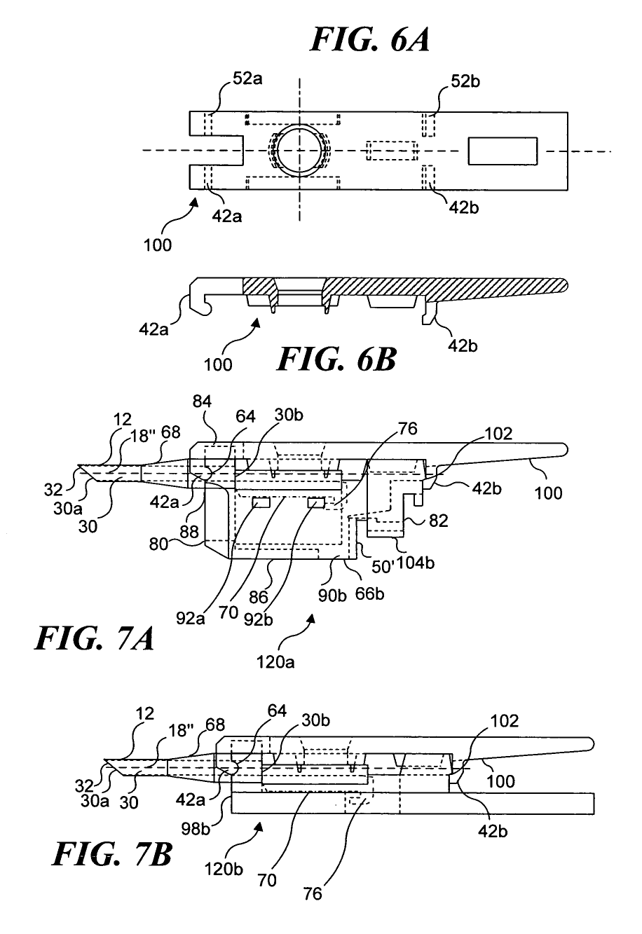

Turning now to FIG. 6A, in a preferred embodiment, package cover 100 includes a first prong 42a and a second prong 52a. Both the first and second prongs are disposed at the distal end of the package cover and the first prong is disposed inwardly and proximate to the side of an outer surface of the cartridge, while the second prong is disposed inwardly and on an opposite side of the outer surface from where the first prong is disposed. The first prong and second prong snap into housing notch 64 of cartridge housing 66b, as shown in FIG. 7A. Similarly the first prong and the second prong can snap into housing notch 64 of package base 98b, as shown in FIG. 7B.

Package cover 100 also includes third prong 42b and fourth prong 52b disposed at the proximal end of the package cover. The third and fourth prongs are disposed inwardly and proximate to the side of the outer surface of the IOL cartridge and snap into an indentation 102 formed on the proximal end of the cartridge housing 66b and package base 98b, as shown in FIGS. 7A and 7B. Those skilled in the art will realize that there are many alternative configurations useful for securing the package cover to cartridge housing 66b, 66c, 66d, and package base 98b, and this invention is not limited to the embodiment disclosed for securing the package cover, as discussed above.

Before turning to the steps for securing the IOL cartridge into its seated position, note that FIG. 8A shows housing 66b ready to be attached to the distal portion of IOL inserter 10b. As shown in FIG. 8B, the cartridge housing has been attached to IOL inserter 10b, preferably with a snap attachment 104a that engages an attachment point 104b. Also, the IOL cartridge is shown in its initial position. As described above, although not shown in FIG. 8B, inserter 10b will typically be preassembled and shipped from a supplier with package cover 100 attached, as shown in FIG. 7A.

Once package cover 100 has been removed from cartridge 12 and cartridge housing 66b, first flange 34 and second flange 36 (FIG. 4A) are then rotated towards each other, thereby folding the IOL and as described above, creating second internal bore 48. Both prongs of the stabilizer (not shown) are removed from first opening 92a and first opening 94a (not shown) and from second opening 92b and second opening 94b (not shown). Removal of the stabilizer enables cartridge 12 to be pressed downwardly into the cartridge housing (cartridge receiving opening 88 is sized to enable this movement), towards the base portion of the cartridge housing, until first notch 124a engages housing protrusion 122a (FIG. 5D) and second notch 124b engages housing protrusion 122b (FIG. 5D), thereby ensuring that the cartridge does not inadvertently move upwardly and out of the cartridge housing. IOL cartridge 12 has thus been moved from its initial position to its seated position. The longitudinal axis of IOL push rod 14 is now aligned with the longitudinal axis of cartridge 12 so that a folded IOL can be forced outwardly from the cartridge and into an eye of a patient by manually advancing the push rod. Also, as shown in FIG. 5D, the first flange and the second flange are now in sliding contact with the housing in order to prevent rotational movement of the cartridge about longitudinal axis 18″.

As illustrated in FIG. 13A and FIG. 13B, after a folded IOL has been forced outwardly from the cartridge and into an eye of a patient by manually advancing IOL push rod 14a, a user has the option of manipulating IOL push rod 14a within cartridge 12a so as to grasp a haptic of an IOL, to maneuver the IOL into a desired position and/or orientation within the lens capsule of the patient's eye. The additional ability to use the IOL inserter to also maneuver the IOL within the lens capsule is advantageous, since the user, typically an ocular surgeon, will already have the IOL inserter inserted through the small incision within the eye and thus, will not have to withdraw the IOL inserter and then reinsert another surgical instrument through the small incision and insert a separate IOL manipulator in order to position the IOL precisely within the lens capsule of the eye.

Thus, FIG. 13A illustrates a typical position of push rod 14a relative to cartridge 12a after the push rod has forced an IOL outwardly from the cartridge. The user can now manipulate the IOL inserter so that proximal haptic 54b of the IOL is engaged by recess 78 of the cartridge. Then, as illustrated in FIG. 13B, the user retracts IOL push rod 14a into cartridge 12a manually (or optionally with the aid of a spring) until the haptic is grasped between notch 26 and recess 78 as shown in FIG. 13B. Once the haptic is thus grasped, the user may guide the haptic to position the IOL in a desired orientation/position within the lens capsule of the patient's eye. The inserter is then disposed of in a medical waste repository.

Alternatively, the haptic may be grasped with a suitably configured distal end of the push rod without the aid of the cartridge. For example, instead of notch 26, a cavity (not shown) may be formed in the distal end of the push rod such that the haptic can be encompassed inside of the cavity and thus maneuvered to orient the IOL as required. The push rod is then manipulated to release the haptic from the cavity, once the IOL is in the desired position.

FIG. 5C and FIG. 5D illustrate a cartridge housing 66b with the flanges of the IOL cartridge shown in a closed state and IOL cartridge 12 disposed in its seated position. IOL inserter 10b is not shown in these Figures.

In the alternative, if IOL inserter 10a is being used, then cartridge 12 is inserted into the first embodiment of the cartridge housing, as described above, i.e., into cartridge housing 66a as shown in FIG. 1A. Locking element 76 is then disposed in housing groove 90a, and cartridge 12 is slid distally along longitudinal axis 18′″. The locking element is further secured in housing groove 90a, which is disposed along the base portion of cartridge housing 66a and ensures that the longitudinal axis of the cartridge is aligned with the longitudinal axis of the inserter so that the IOL is freely advanced from the IOL cartridge into a patient's eye.

### Third Embodiment of Cartridge Housing

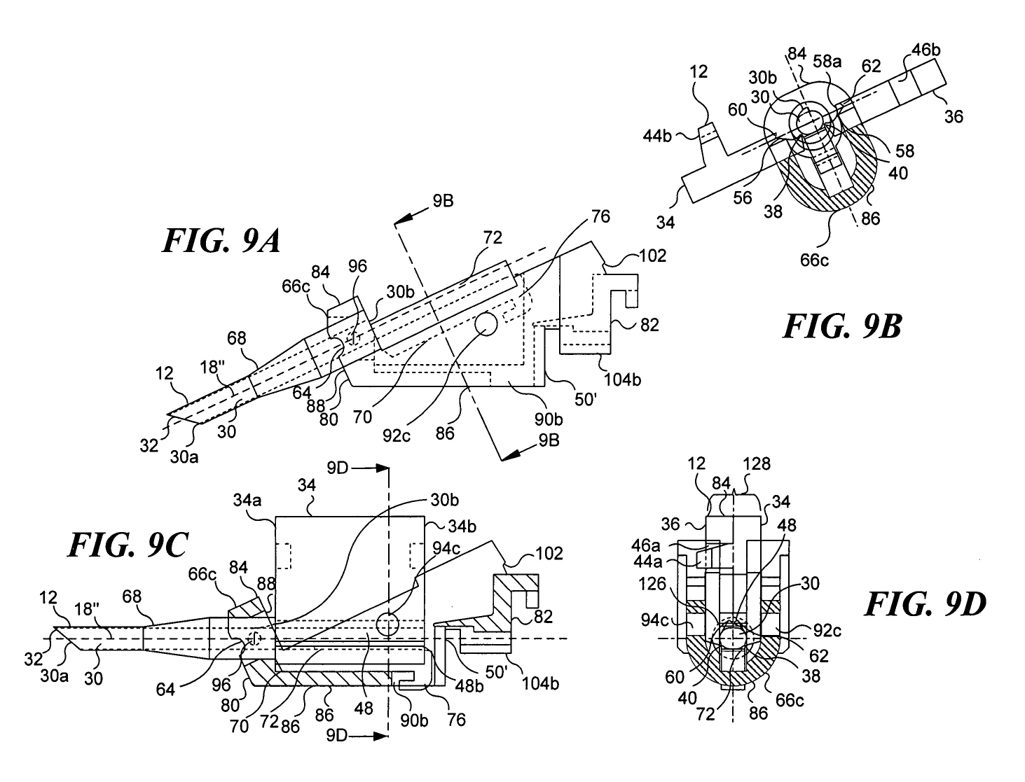

FIG. 9A, FIG. 9B, FIG. 9C, and FIG. 9D illustrate a cartridge housing 66c that can be attached to the distal end of IOL inserter 10c, as shown in FIG. 10B, and FIG. 10C, and as shown prior to attachment in FIG. 10A. Cartridge housing 66c is the third of four preferred embodiments of the cartridge housing.

Cartridge housing 66c is similar to the second embodiment of the cartridge housing, in that it also includes distal end 80, proximal end 82, top portion 84, base portion 86, cartridge receiving opening 88, housing push rod opening 50′, and housing groove 90b. In addition, cartridge housing 66c also includes a pivot point 96 disposed approximately midway between the top portion and the base portion of the distal end of cartridge housing 66c, so that the IOL cartridge is pivotal within the cartridge receiving opening. In addition, FIG. 9A illustrates a first opening 92c that is disposed on the outer surface of the cartridge housing between the top portion and the base portion. There is also a corresponding second opening 94c disposed on the opposite outer surface of the cartridge housing, between the top portion and the base portion relative to where first opening 92c is disposed (see FIG. 9D). Similar to cartridge housing 66b, a stabilizer (not shown) can be inserted through first opening 92c and corresponding second opening 94c to prevent the IOL cartridge from pivoting about pivot point 96 and thus moving from an initial position to a seated position when such movement is not intended. As in the second embodiment of the cartridge housing, package cover 100 (FIG. 6A) is engaged between housing notch 64 and indentation 102 to hold the open cartridge and IOL in place before use.

Next, as shown in FIG. 10B, cartridge housing 66c is attached to IOL inserter 10c, preferably with a snap attachment 104a that engages an attachment point 104b. First flange 34 and second flange 36 (FIG. 4A) are rotated towards each other, thereby folding the IOL, and as described above, creating second internal bore 48. The stabilizer (not shown) is then removed from first opening 92c and corresponding opening 94a. Removal of the stabilizer enables cartridge 12 to be pivoted towards the base portion of the cartridge housing until a securing element (not shown) on the cartridge housing engages the first protrusion and the second protrusion. IOL cartridge 12 has thus now been pivoted from its initial position to its seated position, and the longitudinal axis of push rod 14 is aligned with the longitudinal axis of cartridge 12 so that a folded IOL can be forced outwardly from the cartridge and into a patient's eye.

FIG. 9C and FIG. 9D illustrate cartridge housing 66c with the flanges of the IOL cartridge in a closed state and IOL cartridge 12 disposed in its seated position and do not include IOL inserter 10c.

Optionally, IOL push rod 14a and cartridge 12a can be used with cartridge housing 66c in order to manipulate the forcibly expelled IOL into a desired orientation or position within the lens capsule, as described above in conjunction with cartridge housing 66b.

### Fourth Embodiment of Cartridge Housing

FIGS. 11A, 11B, 11C, and 11D illustrate the fourth preferred embodiment of a cartridge housing 66d that can be attached to the distal end of IOL inserter 10d, as shown in FIGS. 12A, 12B, and 12C. Like the second and third embodiments of the cartridge housing, cartridge housing 66d includes distal end 80, proximal end 82, top portion 84, base portion 86, cartridge receiving opening 88, housing push rod opening 50′, housing groove 90b, pivot point 96, and first opening 92c. However, in order to provide for a better position for use by an ocular surgeon, cartridge housing 66d is configured to be attached to an IOL inserter such that it forms a desired angle relative to cartridge housing 66d. The method of attaching cartridge housing 66d to inserter 10d as shown in FIG. 12B is the substantially the same as that used for attaching cartridge housing 66c to inserter 10c. However, although cartridge housing 66d and IOL cartridge 12 are disposed in a seated position and are ready to be used in a surgical procedure, as shown in FIG. 12C, unlike the second and third embodiments of the cartridge housing, where the longitudinal axis of push rod 14 and the longitudinal axis of the IOL inserter are aligned with the longitudinal axis of cartridge 12, as seen in FIG. 8C and FIG. 10C, the longitudinal axis of inserter 10d is disposed such that it forms predefined angle 106 with the longitudinal axis of cartridge 12, as shown in FIG. 12C. Thus, the longitudinal axis of the first internal bore and the second internal bore (i.e., the longitudinal axis of the cartridge) are not aligned with the longitudinal axis of the IOL inserter in this embodiment. A flexible push rod must be used in this configuration, since it must be capable of bending as it advances around the curve caused by providing the predefined angle between the IOL cartridge and the IOL inserter. As in the second and third embodiments of the cartridge housings, package cover 100 (FIG. 6A) is engaged between housing notch 64 and indentation 102 to hold open cartridge 12 and unfolded IOL 28a in place before use of the IOL.

FIG. 11C and FIG. 11D illustrate cartridge housing 66d with the flanges of IOL cartridge 12 in a closed state and IOL cartridge 12 disposed in its seated position; inserter 10d is not shown in these Figures. Just as explained above, push rod 14a and cartridge 12a can also be used with cartridge housing 66d in order to manipulate the forcibly expelled IOL into a desired orientation or position within the lens capsule.

Although the present invention has been described in connection with the preferred form of practicing it and modifications thereto, those of ordinary skill in the art will understand that many other modifications can be made to the present invention within the scope of the claims that follow. Accordingly, it is not intended that the scope of the invention in any way be limited by the above description, but instead be determined entirely by reference to the claims that follow.

1. An intraocular lens push rod that is configured for use with an intraocular lens inserter and employed to controllably advance an intraocular lens disposed inside an intraocular lens cartridge along a longitudinal axis of the intraocular lens cartridge, the push rod being configured with a proximal end against which a force is applied to advance an intraocular lens along the longitudinal axis, and a distal end contoured to contact a substantial portion of a perimeter of a proximal end of an intraocular lens disposed inside an intraocular lens cartridge, such that the force is more evenly applied by the push rod to advance an intraocular lens along the longitudinal axis of an intraocular lens cartridge.

2. The intraocular lens push rod of claim 1, wherein the distal end of the push rod includes a groove, said groove being disposed on an outer surface of the push rod and sized to accommodate a haptic of an intraocular lens.

3. The intraocular lens push rod of claim 1, wherein a contour of a leading edge of the distal end is shaped to substantially correspond to a proximal end of a folded intraocular lens.

4. The intraocular lens push rod of claim 3, wherein the distal end of the push rod is sized so that an outer surface of the distal end of the push rod is in sliding contact with a substantial portion of an inner surface of an intraocular lens cartridge, to prevent damage of the intraocular lens and to prevent the distal end of the push rod being advanced past a proximal end of an intraocular lens without applying the force thereto.

5. The intraocular lens push rod of claim 1, wherein a trailing edge of the distal end includes a notch sized to engage a haptic of an intraocular lens between the notch and a recess formed in a distal end of an intraocular lens cartridge, to enable an intraocular lens to be maneuvered inside a lens capsule of a patient's eye.

6. The intraocular lens push rod of claim 1, wherein the distal end is shaped to engage an intraocular lens cartridge in a predefined rotational orientation relative to a longitudinal axis of an intraocular lens cartridge.

7. Apparatus for inserting an intraocular lens into a lens capsule and grasping a haptic of the intraocular lens to enable the intraocular lens to be manipulated within the lens capsule, comprising a push rod having a distal end that is sized to advance an intraocular lens through an internal bore of a cartridge in which an intraocular lens is disposed before being advanced into a lens capsule, a surface of the push rod adjacent to its distal end including a notch sized and configured to engage a haptic of an intraocular lens, to enable an intraocular lens to be manipulated after being expelled into a lens capsule.

8. The apparatus of claim 7, further comprising a cooperating surface of a distal end of the cartridge that is used to grasp a haptic of an intraocular lens between said surface and the notch, while manipulating an intraocular lens within a lens capsule.

9. An intraocular lens cartridge system that is configured for use with an intraocular lens inserter and in which an intraocular lens is disposed prior to insertion through a cartridge and into a lens capsule in an eye of a patient, comprising a housing configured to enclose at least a portion of the cartridge, said housing including:
(a) a cartridge receiving opening disposed proximate to a distal end of the housing; said cartridge receiving opening being adapted to receive at least a distal portion of the cartridge and engage the cartridge within the housing in a first position and enable movement of the cartridge within the housing to a second position to secure the cartridge to the housing; and
(b) a push rod opening disposed proximate to a proximal end of the housing; said proximal end being adapted to attach to a distal end of an intraocular lens inserter, said push rod opening being adapted to receive an intraocular lens push rod.

10. The intraocular lens cartridge system of claim 9, wherein the housing further comprises a first protrusion and a second protrusion disposed between a top portion and a base portion of the housing, said first protrusion and said second protrusion being configured to respectively engage a first notch and a second notch included on the cartridge and to align the longitudinal axis of the cartridge with the longitudinal axis of an intraocular lens inserter such that an intraocular lens is readily advanced from the cartridge and into an eye.

11. The intraocular lens cartridge system of claim 9, wherein a portion of the housing disposed between a top portion and a base portion thereof is in sliding contact with a first flange and a second flange of the cartridge.

12. The intraocular lens cartridge system of claim 9, wherein the cartridge comprises:
(a) a distal portion, including a first internal bore with a proximal end and a distal end, the distal end having an opening through which a folded intraocular lens is ejected into an eye;
(b) a lens loading portion having a central groove extending longitudinally;
(c) a first flange coupled to one side of the lens loading portion by a first hinge that extends longitudinally along one side of the central groove; and
(d) a second flange coupled to an opposite side of the lens loading portion by a second hinge that extends longitudinally along an opposite side of the central groove from the first hinge, the first hinge and the second hinge respectively enabling rotation of the first flange and the second flange towards each other, wherein prior to their rotation, the first flange and the second flange cooperate with the lens loading portion to define a lens support region on which an unfolded intraocular lens is disposed, and upon being rotated toward each other, the first flange and the second flange cause an intraocular lens on the lens support region to be folded in preparation for its insertion through the first internal bore of the distal portion.

13. For use with an intraocular lens inserter, a housing for an intraocular lens cartridge employed for insertion of an intraocular lens into an eye of a patient, comprising:
(a) a distal end having an opening sized to enable a distal end of an intraocular lens cartridge to be inserted therethrough; and
(b) a cartridge receiving portion proximal to the opening and configured and sized to receive an intraocular lens cartridge in a first orientation relative to a longitudinal axis of the housing, so that in the first orientation, a distal end of an intraocular lens cartridge is readily inserted through the opening, and then pivoted through an angle to a second orientation relative to the longitudinal axis, and in the second orientation, an intraocular lens cartridge is thus disposed within the housing for use with an intraocular lens inserter.

14. For attachment to an intraocular lens inserter, a housing for an intraocular lens cartridge that is employed for insertion of an intraocular lens into a lens capsule in an eye of a patient, comprising:
(a) a coupling portion that is adapted and configured to connect with a distal end of an intraocular lens inserter; and
(b) a cartridge supporting structure that supports an intraocular lens cartridge while an intraocular lens disposed therein is advanced into a lens capsule of an eye, said coupling portion being connected to the cartridge supporting structure at a predefined angle so that a longitudinal axis of an intraocular lens cartridge supported by the cartridge supporting structure forms a desired angle with a longitudinal axis of an intraocular lens inserter to which the housing is connected.

15. For use with an intraocular lens inserter employed for insertion of an intraocular lens into an eye of a patient, apparatus for maneuvering an intraocular lens after it has been inserted into a lens capsule of an eye, comprising a cartridge that includes a distal end, the distal end having an opening through which a folded intraocular lens is ejected into a lens capsule, the cartridge including a recess disposed adjacent to the distal end of the cartridge for engaging a haptic of an intraocular lens to enable manipulation of an intraocular lens within a lens capsule of an eye.

16. The apparatus of claim 15, further comprising a push rod acting in cooperation with the recess of the cartridge and having a distal end that is sized to advance an intraocular lens through the opening of the cartridge, a surface of the push rod adjacent to its distal end including a notch sized and configured to engage a haptic of an intraocular lens between the notch and the recess of the cartridge.

17. An intraocular lens cartridge system that is configured for use with an intraocular lens inserter and in which an intraocular lens is disposed prior to insertion into an eye of a patient, comprising a cartridge that includes:
(a) a first internal bore through which a folded intraocular lens is ejected into an eye;
(b) a central lens loading portion including a first flange and a second flange coupled to the central lens loading portion; and
(c) wherein the first flange and the second flange cooperate with the lens loading portion to form a second internal bore that is free of any obstruction that can interfere with advancing an intraocular lens through the second internal bore and into the first internal bore.

18. An intraocular lens cartridge system that is configured for use with an intraocular lens inserter and in which an intraocular lens is disposed prior to insertion into an eye of a patient, comprising a cartridge that includes:
(a) a distal portion defining a first internal bore with a proximal end and a distal end and an outer dimension of the proximal end, the distal end having an opening through which a folded intraocular lens is ejected into an eye; and
(b) a lens loading portion including a first flange and a second flange coupled to the lens loading portion, wherein after the first flange and the second flange have been moved toward and into contact with each other, a combined thickness of the first flange and the second flange is at least equal to the outer dimension of the proximal end of the first internal bore.

19. An intraocular lens cartridge system that is configured for use with an intraocular lens inserter and in which an intraocular lens is pre-loaded prior to shipping to an end user, comprising a cartridge that includes:
(a) a distal portion that includes a first internal bore with a proximal end and a distal end, the distal end having an opening through which a folded intraocular lens is ejected into an eye;
(b) a lens loading portion extending longitudinally; and
(c) a first flange and a second flange that cooperate with the lens loading portion to define a lens support region on which an unfolded intraocular lens is preloaded, wherein upon being rotated toward each other, the first flange and the second flange cause a preloaded intraocular lens disposed on the lens support region to be folded in preparation for its insertion through the first internal bore of the distal portion.

20. An intraocular lens cartridge system that is configured for use with an intraocular lens inserter and in which an intraocular lens is disposed prior to insertion into an eye of a patient, comprising a cartridge that includes:
(a) a distal portion, including a first internal bore with a proximal end and a distal end, the distal end having an opening through which a folded intraocular lens is ejected into an eye;
(b) a lens loading portion having a central groove extending longitudinally; and
(c) a locking element disposed on the cartridge, spaced apart from the distal portion, and configured to retain the cartridge in one of:
(i) an intraocular lens inserter; and
(ii) a housing that is coupled to an intraocular lens inserter.

21. The intraocular lens cartridge system of claim 20, wherein the housing comprises:
(a) a cartridge receiving opening disposed proximate to a distal end of the housing; said cartridge receiving opening being adapted to receive at least a distal portion of the cartridge and engage the cartridge within the housing; and
(b) a push rod opening disposed proximate to a proximal end of the housing; said proximal end adapted such that the housing is attachable to a distal end of an intraocular lens inserter, said push rod opening being adapted to receive an intraocular lens push rod.

22. An intraocular lens cartridge system that is configured for use with an intraocular lens inserter and a cartridge in which an intraocular lens is disposed prior to insertion through the cartridge and into an eye of a patient, comprising:
(a) a distal portion, including a first internal bore with a proximal end and a distal end, the distal end having an opening through which a folded intraocular lens is ejected into an eye;
(b) a lens loading portion having a central groove extending longitudinally;
(c) a first flange coupled to one side of the lens loading portion by a first hinge that extends longitudinally along one side of the central groove; and
(d) a second flange coupled to an opposite side of the lens loading portion by a second hinge that extends longitudinally along an opposite side of the central groove from the first hinge, the first hinge and the second hinge respectively enabling rotation of the first flange and the second flange towards each other, wherein prior to their rotation, the first flange and the second flange cooperate with the lens loading portion to define a lens support region wherein an unfolded intraocular lens is disposed, and upon being rotated toward each other, the first flange and the second flange cause an intraocular lens on the lens support region to be folded in preparation for its insertion through the first internal bore of the distal portion.

23. The intraocular lens cartridge of claim 22, wherein a leading edge of the distal end of the first internal bore includes a recess sized to cooperate with a push rod in grasping a haptic of an intraocular lens, to enable maneuvering of an intraocular lens inside a lens capsule of a patient's eye.

24. The intraocular lens cartridge of claim 22, wherein the first flange and the second flange each include one of tapered flat and curved surfaces extending longitudinally between proximal and distal ends of the first flange and the second flange and outwardly of the first hinge and the second hinge, respectively; inwardly projecting lips being formed and extending longitudinally along outer edges of the one of tapered flat and curved surfaces and spaced apart from each of the first hinge and the second hinge, said lips being sized to engage opposite sides of an intraocular lens that is supported on the lens support region, so that as the first flange and the second flange are rotated toward each other, they form a second internal bore having a distal end and a proximal end, the lips retaining an intraocular lens as it is folded between the first flange and the second flange within the second internal bore.

25. The intraocular lens cartridge system of claim 24, wherein one of the first flange and the second flange engages the other of the first flange and the second flange as they are rotated toward each other and into contact with each other about the first hinge and the second hinge.

26. The intraocular lens cartridge system of claim 24, wherein the distal end of the second internal bore is in communication with the proximal end of the first internal bore and both are tapered asymmetrically to cause one side of a folded intraocular lens to overlap an opposite side thereof, as the intraocular lens is advanced through the second internal bore and the first internal bore.

27. The intraocular lens cartridge system of claim 24, wherein when the first flange and the second flange are folded into contact with each other, the lips do not protrude into the second internal bore, leaving it free of any obstruction that can interfere with advancing an intraocular lens and a push rod through the second internal bore and into the first internal bore.

28. The intraocular lens cartridge system of claim 22, further comprising one of:
(a) a locking element disposed adjacent to the proximal end of the lens loading portion, for removably retaining the cartridge in an intraocular lens inserter; and
(b) a first notch and a second notch disposed on the first flange and the second flange, respectively, for retaining the cartridge in a housing adapted to couple to an intraocular lens inserter.

29. The intraocular lens cartridge system of claim 22, further comprising a housing configured to enclose at least a portion of the cartridge, said housing having a distal end, a proximal end, a top portion and a base portion, said housing including:
(a) a cartridge receiving opening disposed proximate to the distal end of the housing; said cartridge receiving opening being adapted to receive at least the distal portion of the cartridge; and
(b) a push rod opening disposed proximate to the proximal end of the housing; said push rod opening being adapted to receive an intraocular lens push rod.

30. The intraocular lens cartridge system of claim 29, further comprising a groove disposed in the base portion of the housing, said groove being configured to accommodate a locking element included on the intraocular lens cartridge.

31. The intraocular lens cartridge system of claim 29, further comprising a first protrusion and a second protrusion disposed between the top portion and the base portion of the housing, said first protrusion and said second protrusion being configured to respectively secure a first notch and a second notch included on the intraocular lens cartridge and to align the longitudinal axis of the intraocular lens cartridge with the longitudinal axis of an intraocular lens inserter such that an intraocular lens is readily advanced from the intraocular lens cartridge and into an eye.

32. The intraocular lens cartridge system of claim 29, further comprising a first opening disposed on the housing between the top portion and the base portion and a second opening disposed opposite the first opening, such that a stabilizer inserted into the first opening and the second opening is disposed substantially perpendicular to the longitudinal axis of the cartridge to initially prevent movement of the intraocular cartridge towards the base portion of the housing.

33. The intraocular lens cartridge system of claim 29, wherein the cartridge receiving opening enables movement of the intraocular lens cartridge between the top portion and the base portion to seat the intraocular lens cartridge within the housing after the first flange and the second flange have been rotated toward and into contact with each other.

34. The intraocular lens cartridge system of claim 29, wherein the intraocular lens cartridge is attachable to the housing and pivotal within the cartridge receiving opening from an initial position in which the first flange and the second flange are not yet rotated toward and into contact with each other, to a seated position within the cartridge receiving opening after the first flange and the second flange have been rotated toward and into contact with each other, so that in the seated position, the longitudinal axis of the cartridge is aligned with the longitudinal axis of an inserter and an intraocular lens is readily advanced from the intraocular lens cartridge and into an eye.

35. The intraocular lens cartridge system of claim 29, wherein the housing is configured and adapted to be mounted to an intraocular lens inserter so that a longitudinal axis of the distal end of the housing and of the intraocular lens cartridge is not aligned with a longitudinal axis of an intraocular lens inserter engaged by the housing when the intraocular lens cartridge has been pivoted to the seated position, and so that the longitudinal axis passing through the first internal bore and the second internal bore of the intraocular lens cartridge forms a predefined angle with the longitudinal axis of an intraocular lens inserter.

36. The intraocular lens cartridge system of claim 29, further comprising a cover that overlies an intraocular lens disposed on the lens loading portion of the intraocular lens cartridge and removably secured to one of a base and the housing, said cover securing an intraocular lens in place on the lens loading portion and protecting the intraocular lens until the cover is removed to enable the intraocular lens cartridge to be used to insert an intraocular lens into an eye.

37. The intraocular lens cartridge system of claim 29, wherein a portion of the housing disposed between the top portion and the base portion thereof is in sliding contact with the first flange and the second flange.

38. A method for folding an intraocular lens disposed in an intraocular lens cartridge into a folded state in which one side of the intraocular lens overlaps an opposite side, as the intraocular lens is advanced through the intraocular lens cartridge and into an eye, comprising the steps of:
(a) with an intraocular lens disposed between opposite flanges that are rotatable toward each other about two spaced apart hinges, rotating the two flanges toward each other, causing the one side and the opposite side of the intraocular lens that is initially unfolded, to be folded toward each other and forming a bore through which the intraocular lens can be advanced after being thus folded, said bore having an asymmetrically tapered shape; and
(b) forcing the lens that is folded to be displaced distally down the bore, the asymmetrically tapered shape of the bore first forcing said one side of the lens to be folded into the bore, and further distally down the bore, next forcing the other side of the lens to increasingly overlap the one side of the lens, as the intraocular lens moves distally through the bore.

39. The method of claim 38, further comprising the step of employing a push rod to displace the intraocular lens distally down the bore.

40. A method of grasping and manipulating an intraocular lens that has been forcibly expelled from a cartridge and into a lens capsule of an eye such that the intraocular lens may be positioned or oriented as desired within the lens capsule, comprising the steps of:
(a) after the intraocular lens has been expelled into the lens capsule from the cartridge using a push rod of an intraocular lens inserter, grasping a haptic of the intraocular lens between a notch formed in the push rod and a surface of the leading edge of the cartridge; and
(b) maneuvering the haptic of the intraocular lens into a desired position or orientation within the lens capsule of the eye.
